(19) United States
(12) Patent Application Publication
(10) Pub. No.: US 2024/0218453 A1
(43) Pub. Date: Jul. 4, 2024

(54) DIAGNOSTIC AND TREATMENT OF CANCER USING C-MET INHIBITOR

(71) Applicant: APOLLOMICS INC., Foster City, CA (US)

(72) Inventors: Xiaoling ZHANG, Foster City, CA (US); Sanjeev REDKAR, San Ramon, CA (US); Guoliang YU, Incline Village, NV (US)

(21) Appl. No.: 18/556,354

(22) PCT Filed: Apr. 21, 2022
(86) PCT No.: PCT/US2022/025727

§ 371 Date: Oct. 20, 2023

Related U.S. Application Data
(60) Provisional application No. 63/177,941, filed on Apr. 21, 2021.

(51) Int. Cl. C12Q 1/6886; A61K 31/5025; A61K 39/00; A61K 45/06; A61P 35/00; C07K 16/28

(52) U.S. Cl. CPC C12Q 1/6886; A61K 31/5025; A61K 45/06; A61P 35/00; C07K 16/2863; A61K 2039/505; C12Q 2600/106; C12Q 2600/158

(57) ABSTRACT

The present disclosure provides methods of treating a cancer in a subject. In one embodiment, the method comprises detecting substantially all RNA transcripts expressed in a sample from the subject, thereby measuring expression level of each gene in a whole transcriptome of the sample; determining that the expression level of HGF is greater than at least 95% of the whole transcriptome; determining that the expression level of c-Met is greater than at least 95% of the whole transcriptome; and administering to the subject a c-Met inhibitor.

### CROSS-REFERENCE TO RELATED APPLICATIONS

This application claims priority to U.S. provisional application 63/177,941, filed Apr. 21, 2021, the disclosure of which is incorporated herein by reference.

### FIELD OF THE INVENTION

The present invention generally relates to cancer treatment. In particular, the present invention relates to methods for treating a cancer patient using c-Met inhibitor when the patient has over-expression of HGF and c-Met.

### BACKGROUND

C-Met, also known as the Hepatocyte Growth Factor Receptor, is a receptor tyrosine kinase that regulates a wide range of different cellular signaling pathways, including those involved in proliferation, motility, migration and invasion. Due to its pleotropic role in cellular processes important in oncogenesis and cancer progression, c-Met aberration has been shown to be involved in a variety of malignancies, such as Small Cell Lung Cancer (SCLC) and NSCLC (Olivero et al., Br J Cancer, 74: 1862-8 (1996) and Ichimura et al., Jpn J Cancer Res, 87:1063-9 (1996)). As a result, c-Met has been considered as an important target in anticancer therapy.

Inhibitors specifically against c-Met represent an attractive novel targeted therapeutic approach. For example, the effectiveness of a novel small molecule specific inhibitor of c-Met, SU11274 was first reported by Sattler, et al. (Pfizer; previously Sugen), in cells transformed by the oncogenic Tpr-Met as a model, as well as in SCLC (Sattler, et al., Cancer Res, 63, (17), 5462-9 (2003)). Recently, small molecular inhibitors of c-Met, such as APL-101 (Apollomics, also known as bozitinib, vebreltinib and PLB-1001), capmatilib (Novartis), tepotinib (Merck) and savolitinib (AstraZeneca), have shown promising efficacy in the clinical trials against lung cancers and brain tumors. However, clinical data indicates that many cancer patients are not responsive to c-Met inhibitors and the efficacy of c-Met inhibitors is limited. Therefore, there is an urgent need to develop new methods for treating cancer patients using c-Met inhibitors.

### SUMMARY

The present disclosure in one aspect provides a method for identifying a subject having cancer as likely to respond to treatment with a c-Met inhibitor. In one embodiment, the method comprises: obtaining a sample from the subject; detecting substantially all RNA transcripts expressed in the sample, thereby measuring expression level of each gene in a whole transcriptome of the sample; determining that the expression level of HGF is greater than a first threshold percentage (e.g., at least 95%) of all genes in the whole transcriptome; determining that the expression level of c-Met is greater than a second threshold percentage (e.g., at least 95%) of all genes in the whole transcriptome; determining that the subject is likely to respond to the treatment with a c-Met inhibitor.

In another embodiment, the method for identifying a subject having cancer as likely to respond to treatment with a c-Met inhibitor comprises: obtaining a sample from the subject; detecting RNA transcripts of a set of biomarker genes expressed in the sample, thereby measuring expression level of each gene in the set of biomarker genes of the sample, wherein the set of biomarker genes comprises ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK; determining that the expression level of HGF is greater than a first threshold percentage, for example, at least 75% of the other genes in the set of biomarker genes; determining that the expression level of c-Met is greater than a second threshold percentage, for example, at least 95% of the other genes in the set of biomarker genes; and determining that the subject is likely to respond to the treatment with a c-Met inhibitor.

In another aspect, the present disclosure provides a method of treating a subject having a cancer. In one embodiment, the method comprises: detecting substantially all RNA transcripts expressed in a sample from the subject, thereby measuring expression level of each gene in a whole transcriptome of the sample; determining that the expression level of HGF is greater than a first threshold percentage (e.g., at least 95%) of all genes in the whole transcriptome; determining that the expression level of c-Met is greater than a second threshold percentage (e.g., at least 95%) of all genes in the whole transcriptome; and administering to the subject a therapeutic effective amount of a c-Met inhibitor.

In another embodiment, the method of treating a subject having a cancer comprises: detecting RNA transcripts of a set of biomarker genes expressed in a sample from the subject, thereby measuring expression level of each gene in the set of biomarker genes of the sample, wherein the set of biomarker genes comprises ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK; determining that the expression level of HGF is greater than a first threshold percentage, for example, at least 70% of all other genes in the set of biomarker genes; determining that the expression level of c-Met is greater than a second threshold percentage, for example, at least 95% of all other genes in the set of biomarker genes; and administering to the subject a therapeutic effective amount of a c-Met inhibitor.

In some embodiments, the first or second threshold percentage is at least 96%, 97%, 98% or 99% of the whole transcriptome. In some embodiments, the first or second threshold percentage is at least 75%, 80%, 85%, 90%, 95%, 96%, 97%, 98% or 99% of the set of biomarker genes.

In some embodiments, the method comprises ranking the expression level of each gene in the whole transcriptome or the set of biomarker genes before determining that the expression level of HGF or c-Met is greater than a threshold percentage all genes of the whole transcriptome or the set of biomarker genes.

In some embodiments, the sample does not have a mutation in c-Met gene. In certain embodiments, the sample does not have an oncogenic mutation in c-Met gene. In certain embodiments, the oncogenic mutation in c-Met gene is a point mutation that generates an alternative splicing encoding a shorter protein that lacks exon 14, which encodes for juxtamembrane domain of c-Met; a point mutation in the kinase domain that renders the enzyme constitutively active; or a Y1003 mutation that inactivate the Cb1 binding site leading to constitutive c-Met expression. In certain embodiments, the mutation in c-Met gene generates a c-Met fusion gene. In certain embodiments, the mutation in c-Met gene is disclosed in PCT/CN2020/094824, which is incorporated herein through reference.

In some embodiments, the cancer is selected from the groups consisting of a lung cancer, a melanoma, a renal cancer, a liver cancer, a myeloma, a prostate cancer, a breast cancer, a colorectal cancer, a pancreatic cancer, a thyroid cancer, a schwannoma, a hematological cancer, a leukemia and a non-Hodgkin's lymphoma. In some embodiments, the cancer is a non-small cell lung cancer (NSCLC), renal cell carcinoma or hepatocellular carcinoma.

In some embodiments, the c-Met inhibitor is selected from the group consisting of crizotinib, cabozantinib, APL-101 (aka CBT-101, PLB1001, bozitinib, vebreltinib), SU11274, PHA665752, K252a, PF-2341066, AM7, JNJ-38877605, PF-04217903, MK2461, GSK1363089 (aka XL880, foretinib), AMG458, tivantinib (aka ARQ197), INCB28060 (aka INC280, capmatinib), E7050, BMS-777607, savolitinib (aka volitinib), tepotinib, HQP-8361, merestinib, ARGX-111, onartuzumab, rilotumumab, emibetuzumab, and XL184.

In some embodiments, the c-Met inhibitor comprising a compound of the following formula

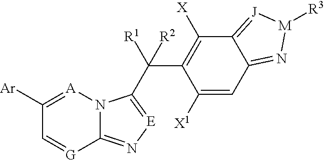

wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or CH;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl.

In some embodiments, the compound is APL-101, which has the following formula

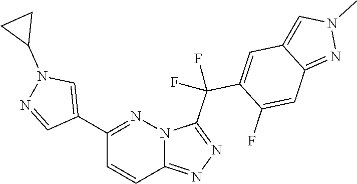 [SMILES: Cn1cc2cc(C(F)(F)c3nnc4ccc(-c5cnn(C6CC6)c5)nn34)c(F)cc2n1]

In some embodiments, the c-Met inhibitor is an anti-c-Met antibody.

### BRIEF DESCRIPTION OF DRAWING

FIG. 1 shows c-MET over-expression of patient 003-019 determined by IHC.

FIG. 2 shows that the gene expression levels of both MET and HGF are aberrantly high in patient 003-019 whole-transcriptome.

FIG. 3 shows that the gene expression levels of both MET and HGF are aberrantly high in patient 003-019 compared to the dynamic range surveyed by 93 unique tumor types and matched 30 tissue types.

FIG. 4 shows the percentiles of the MET and HGF gene expression ranking among the 40-cancer gene-signatures in SA4097 sarcoma PDX model.

FIG. 5 shows the percentiles of the MET and HGF gene expression ranking among the 40-cancer gene-signatures in KP4 human pancreatic cancer cell line.

FIG. 6 shows the percentiles of the MET and HGF gene expression ranking among the 40-cancer gene-signatures in SA10199 sarcoma PDX model.

FIG. 7 shows that c-Met inhibitors APL-101 and capmatinib inhibited tumor growth in SA4097 sarcoma PDX model.

FIG. 8 shows that c-Met inhibitors APL-101, capmatinib, tepotinib and savolitinib inhibited tumor growth in KP4 human pancreatic cancer cell line xenograft model.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

As used herein, the term “administering” means providing a pharmaceutical agent or composition to a subject, and includes, but is not limited to, administering by a medical professional and self-administering.

As used herein, an “anti-angiogenesis agent” means a substance that reduces or inhibits the growth of new blood vessels, such as, e.g., an inhibitor of vascular endothelial growth factor (VEGF) and an inhibitor of endothelial cell migration. Anti-angiogenesis agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

As used herein, the term “cancer” refers to any diseases involving an abnormal cell growth and includes all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

The term “cancer sample” includes a biological sample or a sample from a biological source that contains one or more cancer cells. Biological samples include samples from body fluids, e.g., blood, plasma, serum, or urine, or samples derived, e.g., by biopsy, from cells, tissues or organs, preferably tumor tissue suspected to include or essentially consist of cancer cells.

The term “c-Met” refers to a proto-oncogene that encodes a protein known as hepatocyte growth factor receptor (HGFR). c-Met protein is composed of the α chain and β chain generated by cleaving a precursor of c-Met (pro c-Met) and forms a dimer by a disulfide linkage. c-Met is a receptor penetrating a cell membrane and the entire a chain and a part of the R chain are present extracellularly (see, e.g., Mark, et al., The Journal of Biological Chemistry (1992) 267:26166-71; Ayumi I, Journal of Clinical and Experimental Medicine (2008) 224:51-55). See also GenBank Accession No: NP-000236.2 for human c-Met and its α chain and β chain. It has been shown that abnormal c-Met activation in cancer correlates with poor prognosis, where aberrantly active c-Met triggers tumor growth, formation of new blood vessels that supply the tumor with nutrients, and cancer spread or other organs.

A “c-Met inhibitor,” as used herein, refers an agent that can suppress the expression or activity of c-Met protein. Examples of c-Met inhibitor include, without limitation crizotinib, cabozantinib, tepotinib, AMG337, APL-101 (aka PLB1001, bozitinib), SU11274, PHA665752, K252a, PF-2341066, AM7, JNJ-38877605, PF-04217903, MK2461, GSK1363089 (aka XL880, foretinib), AMG458, Tivantinib (ARQ197), INCB28060 (aka INC280, capmatinib), E7050, BMS-777607, savolitinib (aka volitinib), HQP-8361, merestinib, ARGX-111, onartuzumab, rilotumumab, emibetuzumab XL184 and compounds disclosed in US20150218171.

The terms “determining,” “assessing,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

As used herein, the term “expression level” of a gene means the amount of product, e.g., RNA or protein, expressed from a gene. In certain embodiment, the expression level of a gene refers to the amount of RNA transcript expressed from that gene. In certain embodiments, the expression level of a gene is measured in a high throughput sequencing assay (e.g., RNA-seq). In such case, each read is first mapped to a reference transcript annotation to determine to which gene the read belongs. The expression level is then quantified by counting the number of reads that mapped to each gene. As longer genes will have more fragments/reads/counts than shorter genes if transcript expression is the same, in some embodiments, the expression level is adjusted by dividing the number of reads by the length of a gene (mRNA). In some embodiments, the expression level is further normalized by per million scaling factor (Transcript Per Million, TPM).

In the present invention, the term “immunomodulator” means a substance that alters the immune response by augmenting or reducing the ability of the immune system to produce antibodies or sensitize cells that recognize and react with the antigen that initiated their production. Immunomodulators may be recombinant, synthetic, or natural preparations and include cytokines, corticosteroids, cytotoxic agents, thymosin, and immunoglobulins. Some immunomodulators are naturally present in the body, and certain of these are available in pharmacologic preparations. In certain embodiments, immunomodulators are modulators of an immune checkpoint. Examples of immunomodulators include, but are not limited to, granulocyte colony-stimulating factor (G-CSF), interferons, imiquimod and cellular membrane fractions from bacteria, IL-2, TL-7, IL-12, CCL3, CCL26, CXCL7, and synthetic cytosine phosphate-guanosine (CpG).

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “photoactive therapeutic agent” means compounds and compositions that become active upon exposure to light. Certain examples of photoactive therapeutic agents are disclosed, e.g., in U.S. Patent Application Publication Serial No. 2011/015223.

As used herein, the term “radiosensitizing agent” means a compound that makes tumor cells more sensitive to radiation therapy. Examples of radiosensitizing agents include misonidazole, metronidazole, tirapazamine, and trans sodium crocetinate.

The terms “responsive,” “clinical response,” “positive clinical response,” and the like, as used in the context of a patient's response to a cancer therapy, are used interchangeably and refer to a favorable patient response to a treatment as opposed to unfavorable responses, i.e., adverse events. In a patient, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. In a population the clinical benefit of a drug, i.e., its efficacy can be evaluated on the basis of one or more endpoints. For example, analysis of overall response rate (ORR) classifies as responders those patients who experience CR or PR after treatment with drug. Analysis of disease control (DC) classifies as responders those patients who experience CR, PR or SD after treatment with drug. A positive clinical response can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of recurrence-free interval (RFI), an increase in the time of survival as compared to overall survival (OS) in a population, an increase in the time of disease-free survival (DFS), an increase in the duration of distant recurrence-free interval (DRFI), and the like. Additional endpoints include a likelihood of any event (AE)-free survival, a likelihood of metastatic relapse (MR)-free survival (MRFS), a likelihood of disease-free survival (DFS), a likelihood of relapse-free survival (RFS), a likelihood of first progression (FP), and a likelihood of distant metastasis-free survival (DMFS). An increase in the likelihood of positive clinical response corresponds to a decrease in the likelihood of cancer recurrence or relapse.

The term “sample” as used herein refers to a biological sample that is obtained from a subject and contains RNA transcripts, genomic DNAs, and/or proteins. Examples of sample include, without limitation, cells, such as cancer cells, tissues, such as biopsy tissue (e.g. biopsied bone tissue, bone marrow, breast tissue, gastrointestinal tract tissue, lung tissue, liver tissue, prostate tissue, brain tissue, nerve tissue, meningeal tissue, renal tissue, endometrial tissue, cervical dittuse, lymph node tissue, muscle tissue, or skin tissue) and paraffin embedded tissues, and bodily fluid, such as blood, plasma, serum, urine, vaginal fluid, uterine or vaginal flushing fluids, plural fluid, ascitic fluid, cerebrospinal fluid, saliva, sweat, tears, sputum, bronchioalveolar lavage fluid, etc. In certain embodiments, the sample can be a biological sample comprising cancer cells. In some embodiments, the sample is a fresh or archived sample obtained from a tumor, e.g., by a tumor biopsy or fine needle aspirate. The sample also can be any biological fluid containing cancer cells. The collection of a sample from a subject is performed in accordance with the standard protocol generally followed by hospital or clinics, such as during a biopsy.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “toxin” means an antigenic poison or venom of plant or animal origin. An example is diphtheria toxin or portions thereof.

As used herein, the term “transcriptome” means the set of all or substantially all RNA transcripts in a sample, e.g., an individual cell or a population of cells. In some embodiments, transcriptome refers to all mRNA transcripts in a sample. In some embodiments, transcriptome includes all protein-coding and non-coding RNA transcripts.

The term “treatment,” “treat,” or “treating” refers to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%, 80%, 90%, or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

### Transcriptome Analysis

The present disclosure in one aspect provides a method for identifying a subject having cancer as likely to respond to treatment with a c-Met inhibitor. In one embodiment, the method comprises: obtaining a sample from the subject; detecting substantially all RNA transcripts expressed in the sample, thereby measuring expression level of each gene in a whole transcriptome of the sample; determining that the expression level of HGF is greater than at least 95% of all genes in the whole transcriptome; determining that the expression level of c-Met is greater than at least 95% of all genes in the whole transcriptome; determining that the subject is likely to respond to the treatment with a c-Met inhibitor.

In another embodiment, the method for identifying a subject having cancer as likely to respond to treatment with a c-Met inhibitor comprises comparing the expression level of HGF and c-Met to a set of biomarker genes that are commonly altered in all cancer patients. In one embodiment, the method comprises: obtaining a sample from the subject; detecting RNA transcripts of a set of biomarker genes expressed in the sample, thereby measuring expression level of each gene in the set of biomarker genes of the sample; determining that the expression level of HGF is greater than at least 75% of the other genes in the set of biomarker genes; determining that the expression level of c-Met is greater than at least 95% of the other genes in the set of biomarker genes; and determining that the subject is likely to respond to the treatment with a c-Met inhibitor. In one embodiment, the set of biomarker genes comprises ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK.

The proto-oncogene c-MET encodes for the receptor tyrosine kinase (RTK) c-Met. Cells of epithelial-endothelial origin widely express c-MET, where it is essential for embryonic development and tissue repair. Hepatocyte growth factor (HGF) is the only known ligand for the c-Met receptor and is expressed mainly in cells of mesenchymal origin. Under normal conditions, c-Met dimerizes and autophosphorylates upon ligand binding, which in turn creates active docking sites for proteins that mediate downstream signaling leading to the activation of the mitogen-activated protein kinase (MAPK), phosphatidylinositol 3-kinase (PI3K)-AKT, v-src sarcoma viral oncogene homolog (SRC), signal transducer and activator of transcription (STAT) signaling pathways. Such activation evokes a variety of pleiotropic biological responses leading to increased cell growth, scattering and motility, invasion, protection from apoptosis, branching morphogenesis, and angiogenesis. However, under pathological conditions improper activation of c-Met may confer proliferative, survival and invasive/metastatic abilities of cancer cells.

Deregulation and the consequent aberrant signaling of c-Met may occur by different mechanisms including gene amplification and activating mutations. It has been reported that c-Met is overexpressed in a variety of carcinomas including lung, breast, ovary, kidney, colon, thyroid, live rand gastric carcinomas. Such overexpression could be the result of transcription activation, hypoxia-induced overexpression, or as a result of c-Met gene amplification. While gene amplification is a frequent genetic alteration of c-Met and has been reported as associated with a poor prognosis in NSCLC, colorectal and gastric cancer, oncogenic mutations on the c-Met gene are rarely found in patients with nonhereditary cancer. Potential oncogenic mutations of c-Met involve mainly point mutations that generate an alternative splicing encoding a shorter protein that lacks exon 14, which encodes for juxtamembrane domain of c-Met; point mutations in the kinase domain that render the enzyme constitutively active; and Y1003 mutations that inactivate the Cb1 binding site leading to constitutive c-Met expression.

In the present disclosure, the inventors have surprisingly found that overexpression of multiple components in the c-Met signaling pathway, e.g., HGF and c-Met, is indicative of a patient's responsiveness to treatment of a c-Met inhibitor. In particular, the overexpression of genes in the c-Met signaling pathway is measured based on transcriptome analysis.

The transcriptome described herein encompasses all the RNA transcripts present in a given organism or biological sample. In certain embodiments, the transcriptome refers to all RNAs in a biological sample. In certain embodiments, the transcriptome refers to all mRNAs in a biological sample. In certain embodiments, the transcriptome refers to all protein-coding RNAs in a biological sample. In certain embodiments, the transcriptome encompasses all protein-coding and non-coding RNAs.

The transcriptome of a biological sample can be measured by proper methods known in the art including without limitation, microarray, a hybridization-based assay, and RNA-seq, a sequencing-based assay.

### Sample Preparation

Any biological sample suitable for conducting the methods provided herein can be obtained from the subject. In certain embodiments, the sample can be further processed by a desirable method for measuring transcriptome.

In certain embodiments, the method of sample preparation comprises isolating or extracting cancer cell (such as circulating tumor cell) from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, the method further comprises isolating the nucleic acid, e.g., RNA from the sample. Various methods of extraction are suitable for isolating the RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001). In certain embodiments, the RNA isolated from the sample can be reverse transcribed to cDNA before subject to microarray or sequencing.

### Microarray

Nucleic acid hybridization assays use probes to hybridize to the target nucleic acid, thereby allowing detection of the target nucleic acid. Microarray is a hybridization-based assay that provides a method for the simultaneous measurement of the levels of large numbers of target nucleic acid molecules, which can be RNA, DNA, cDNA reverse transcribed from mRNA, or chromosomal DNA. As used herein, the target nucleic acids, e.g., RNA or cDNA reverse transcribed from mRNA, can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

### Sequencing Methods

Sequencing methods useful in the measurement of the transcriptome in a biological sample can be high throughput sequencing (next generation sequencing). High throughput sequencing, or next generation sequencing, by using methods distinguished from traditional methods, such as Sanger sequencing, is highly scalable and able to sequence the entire genome or transcriptome at once. High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina© sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the transcriptome described herein is measured by whole transcriptome shotgun sequencing (RNA sequencing). The method of RNA sequencing has been described (see Wang Z, Gerstein M and Snyder M, Nature Review Genetics (2009) 10:57-63; Maher C A et al., Nature (2009) 458:97-101; Kukurba K & Montgomery SB, Cold Spring Harbor Protocols (2015) 2015(11): 951-969).

### Measuring and Ranking of Expression Level

Microarray and high throughput sequencing provide a measurement of the expression level of all (or substantially all, i.e., at least 95%, 96%, 97%, 98%, 99%, 99.9% depending on the sensitivity of the assay) genes in a biological sample.

In a microarray assay, the expression level of a gene can be measured based on the amount of RNA or cDNA hybridized with the probes on the microarray, as represented by the intensity of the dye (e.g., fluorophore) attached to the RNA or cDNA.

In a high throughput sequencing assay (e.g., RNA-seq), each read is first mapped to a reference transcript annotation to determine to which gene the read belongs. The expression level is then quantified by counting the number of reads that mapped to each gene. As longer genes will have more fragments/reads/counts than shorter genes if transcript expression is the same, in some embodiments, the expression level is adjusted by dividing the number of reads by the length of a gene (mRNA) and normalized by per million scaling factor (Transcript Per Million, TPM). In some embodiment, the expression level is adjusted by normalized the number of fragments by the length of exons of a gene per million mapped fragments (fragments per kilobase of exon per million mapped fragments, FPKM).

In some embodiments, after the expression level of each gene in a transcriptome or a set of biomarker genes is measured, the genes are ranked according to their expression level. In some embodiment, the relative expression level of the genes involved in the c-Met signaling pathway, e.g., HGF and c-Met, as compared to the whole transcriptome or the set of biomarker genes is then determined. In some embodiment, a subject is identified as likely responsive to a treatment of c-Met inhibitor if multiple components in the c-Met signaling pathway belong to the most highly expressed genes in the transcriptome or the set of biomarker genes. In some embodiments, a subject is identified as likely responsive to a treatment of c-Met inhibitor if the expression level of both HGF and c-Met in the biological sample is higher than at least 95%, 96%. 97%, 98% or 99% of all the genes in a transcriptome. In some embodiments, a subject is identified as likely responsive to a treatment of c-Met inhibitor if the expression level of HGF is higher than at least 70%, 75%, 80%, 85%, 90%, 95%, 96%, 97%, 98%, or 99% of all the genes in the set of biomarker genes and the expression level of c-Met in the biological sample is higher than at least 95%, 96%. 97%, 98% or 99% of all the genes in the set of biomarker genes.

Treatment with c-Met Inhibitor

In another aspect, the present disclosure provides a method of treating a subject having cancer. In one embodiment, the method comprises: detecting substantially all RNA transcripts expressed in a sample from the subject, thereby measuring expression level of each gene in a whole transcriptome of the sample; determining that the expression level of HGF is greater than a first threshold percentage, for example, at least 95% of all genes in the whole transcriptome; determining that the expression level of c-Met is greater than a second threshold percentage, for example, at least 95% of all genes in the whole transcriptome; and administering to the subject a therapeutic effective amount of a c-Met inhibitor.

A “c-Met inhibitor,” as used herein, refers to an agent that can suppress the expression or activity of c-Met protein. In certain embodiments, c-Met inhibitor is selected from the group consisting of crizotinib, cabozantinib, tepotinib, AMG337, APL-101 (aka PLB1001, bozitinib, vebreltinib), SU11274, PHA665752, K252a, PF-2341066, AM7, JNJ-38877605, PF-04217903, MK2461, GSK1363089 (aka XL880, foretinib), AMG458, tivantinib (aka ARQ197), INCB28060 (aka INC280, capmatinib), E7050, BMS-777607, savolitinib (aka volitinib), HQP-8361, merestinib, ARGX-111, onartuzumab, rilotumumab, emibetuzumab, and XL184.

In some embodiments, the c-Met inhibitor comprises a compound of the following formula

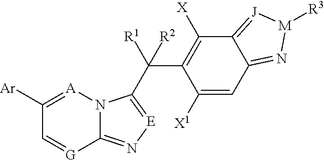

wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆ alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl;

In some embodiments, the c-Met inhibitor is selected from the group consisting of:

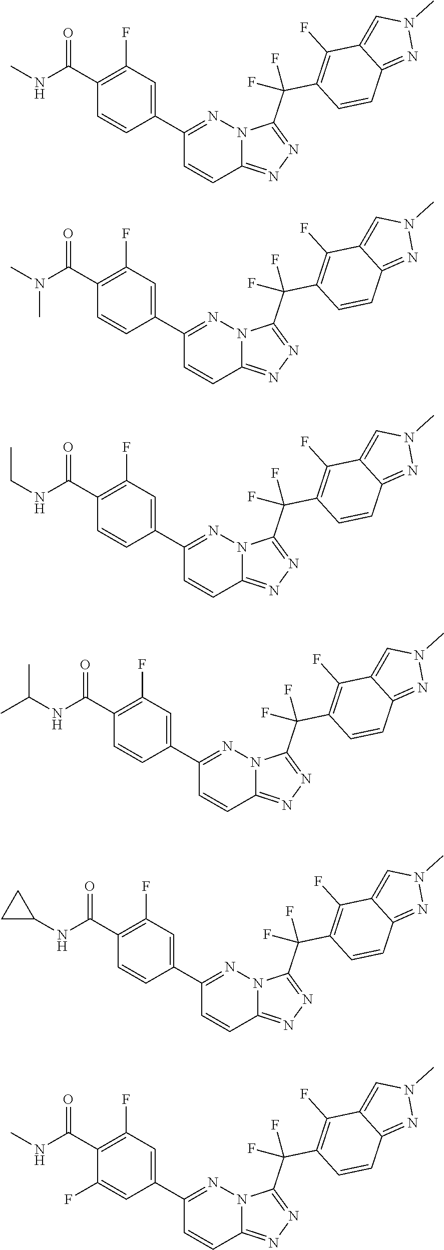

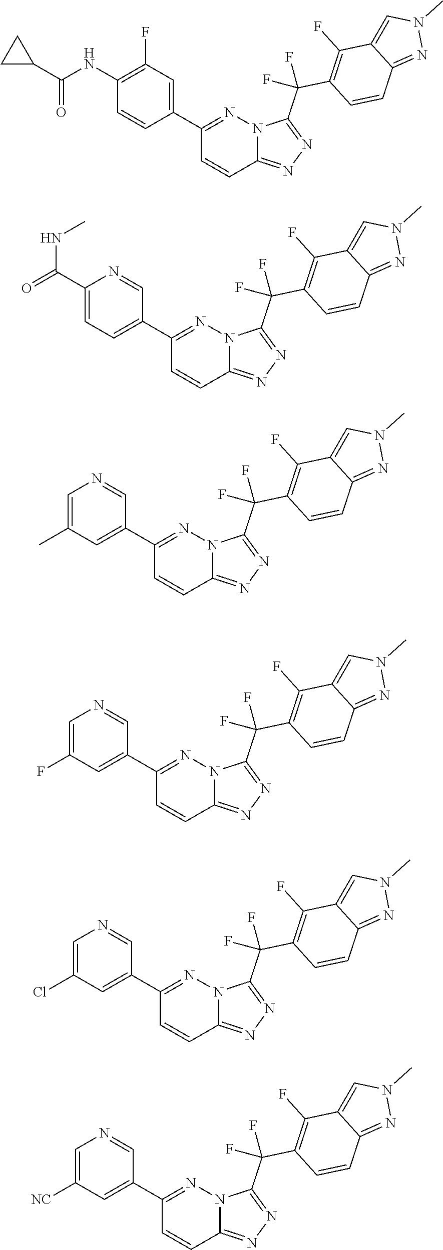

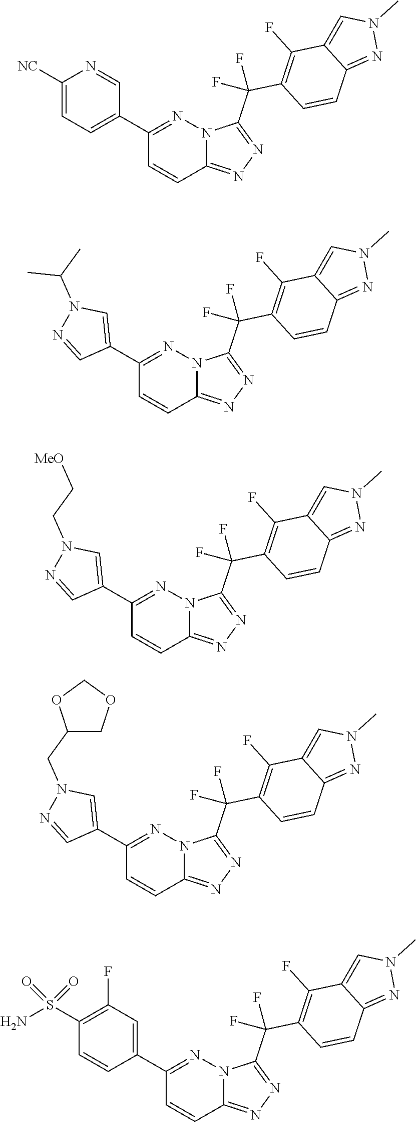

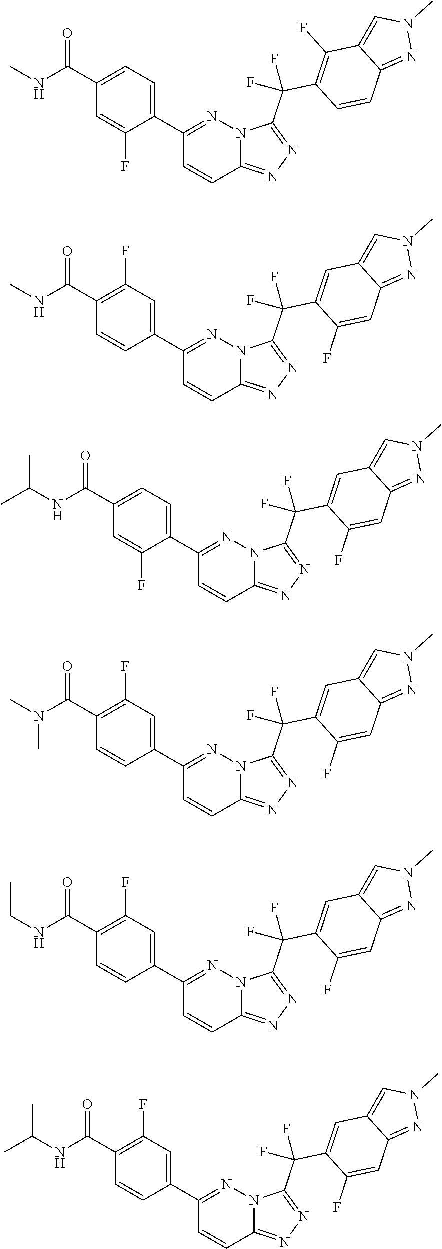

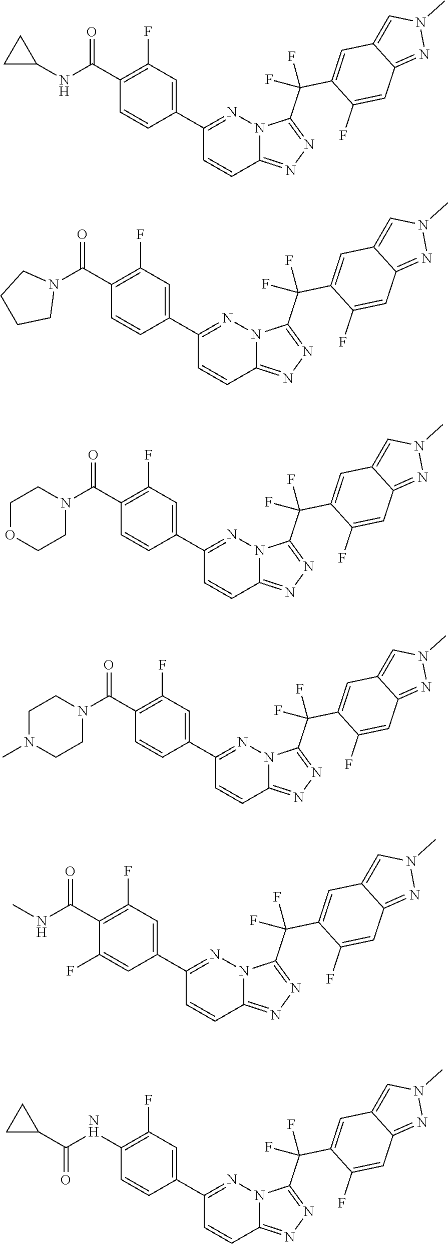

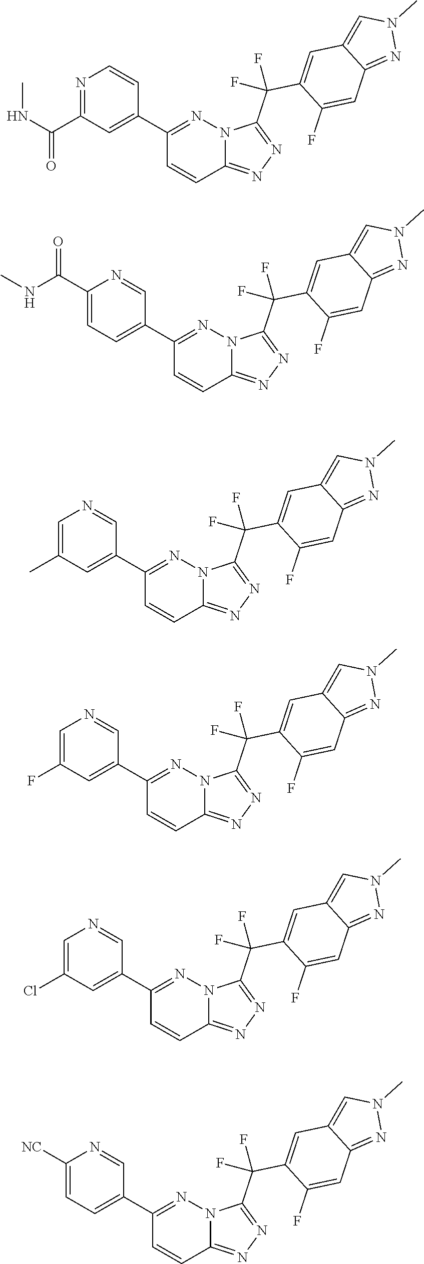

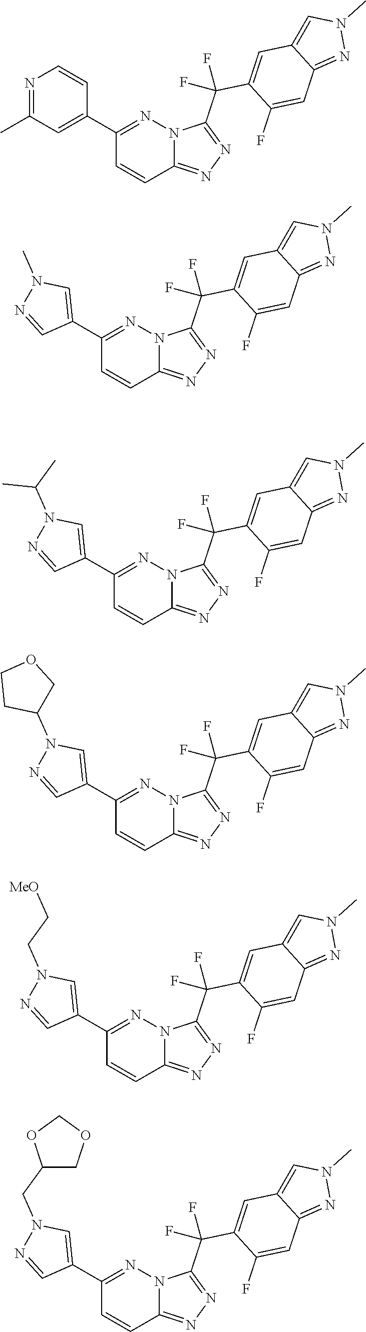

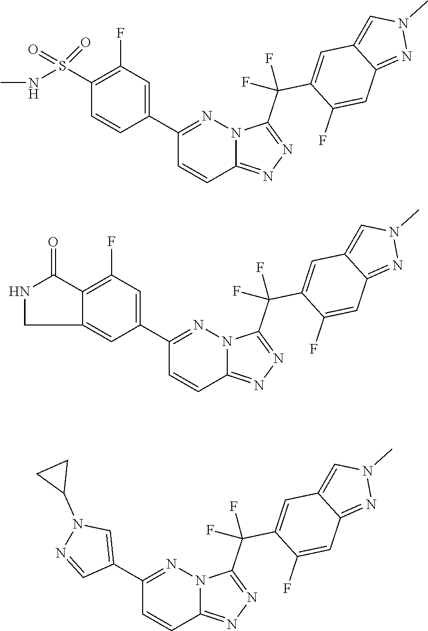

In certain embodiments, c-Met inhibitor is APL-101 (previously named CBT-101, see US20150218171, which is incorporated in its entirety by reference), which has the following formula:

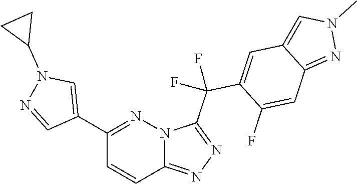 [SMILES: Cn1cc2cc(C(F)(F)c3nnc4ccc(-c5cnn(C6CC6)c5)nn34)c(F)cc2n1]

In certain embodiments, c-Met inhibitor can be formulated with a pharmaceutically acceptable carrier. The carrier, when present, can be blended with c-Met inhibitor in any suitable amounts, such as an amount of from 5% to 95% by weight of carrier, based on the total volume or weight of c-Met inhibitor and the carrier. In some embodiments, the amount of carrier can be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier in a specific embodiment may be determined based on considerations of the specific dose form, relative amounts of c-Met inhibitor, the total weight of the composition including the carrier, the physical and chemical properties of the carrier, and other factors, as known to those of ordinary skill in the formulation art.

The c-Met inhibitor may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the c-Met inhibitor may be administered in conjunction with other treatments. The c-Met inhibitor may be encapsulated or otherwise protected against gastric or other secretions, if desired.

A suitable, non-limiting example of a dosage of the c-Met inhibitor disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. In some embodiments, the dosage of the c-Met inhibitor in human is about 400 mg/day given every 12 hours. In some embodiments, the dosage of the c-Met inhibitor in human ranges 300-500 mg/day, 100-600 mg/day or 25-1000 mg/day. The effective dose of c-Met inhibitor disclosed herein may be administered as two, three, four, five, six or more sub-doses, administered separately at appropriate intervals throughout the day.

In one embodiment, the method further comprises administering at least one additional therapeutic agent selected from the group consisting of a modulator of immune checkpoint, a cytotoxic agent, a toxin, a radionuclide, an immunomodulator, a photoactive therapeutic agent, a radiosensitizing agent, a hormone, an anti-angiogenesis agent, and combinations thereof.

As used herein, the term “immune checkpoint” or “cancer immune checkpoint” refers to a molecule in the immune system that either turns up a signal (i.e., co-stimulatory molecules) or turns down a signal (i.e., inhibitory molecule) of an immune response. In certain embodiments, the immune checkpoint is selected from the group consisting of PD-1, PD-L1, PD-L2, LAG-3, TIM-1, CTLA-4, VISTA, B7-H2, B7-H3, B7-H4, B7-H6, 284, ICOS, HVEM, CD160, gp49B, PIR-B, KIR family receptors, TIM-1, TIM-4, BTLA, SIRPalpha (CD47), CD48, 284 (CD244), B7.1, B7.2, ILT-2, TLT-4, TIGIT and A2aR.

In certain embodiments, the modulator of immune checkpoint is a monoclonal antibody against the immune checkpoint. In certain embodiments, the immune checkpoint is PD-1 or PD-L1. In certain embodiments, the anti-PD-1 antibody is selected from those disclosed in PCT application publication No. WO2016/014688, which is incorporated in its entirety by reference. In certain embodiments, the anti-PD-1 antibody is APL-501 (previously named as CBT-501, see WO2016/014688), GB226 or genolimzumab. In certain embodiments, the anti-PD-L1 antibody is selected from those disclosed in PCT application publication No. WO2016/022630, which is incorporated in its entirety by reference. In certain embodiments, the anti-PD-L1 antibody is APL-502 (previously named as CBT-502, see WO2016/022630) or TQB2450.

Anti-Cancer Agents Other than c-Met Inhibitor

The method of present disclosure also involves, after determining that a subject is not likely to respond to a c-Met inhibitor, administering to the subject an anti-cancer agent other than a c-Met inhibitor. These anti-cancer agents include, without limitation: alkylating agents or agents with an alkylating action, such as cyclophosphamide (CTX; e.g. Cytoxan®), chlorambucil (CHL; e.g. Leukeran®), cisplatin (CisP; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6MP), 6-thiocguanine (6TG), cytarabine (Ara-C), 5-fluorouracil (5-FU), capecitabine (e.g., Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and pactitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin, folinic acid, raltitrexed, and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lornustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, and chlorambucil.

In certain embodiments, an anti-cancer agent other than a c-Met inhibitor is an anti-hormonal agent. As used herein, the term “anti-hormonal agent” includes natural or synthetic organic or peptide compounds that act to regulate or inhibit hormone action on tumors.

Anti-hormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide (2-methyl-N-[4, 20-nitro-3-(trifluoromethyl)phenylpropanamide), commercially available as Eulexin® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

In certain embodiments, an anti-cancer agent other than a c-Met inhibitor is an angiogenesis inhibitor. Anti-angiogenic agents include, for example: VEGFR inhibitors, such as SU-5416 and SU-6668 (Sugen Inc. of South San Francisco, Calif, USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. Avastin™, Genentech, South San Francisco, Calif), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. Vitaxin®); factors such as IFN-alpha (U.S. Pat. Nos. 41,530,901, 4,503,035, and 5,231,176); angiostatin and plasminogen fragments (e.g. kringle 14, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; fik-1 and fit-1 antagonists; anti-angiogenesis agents such as MMP-2 (matrix-metalloprotienase 2) inhibitors and MMP-9 (matrix-metalloprotienase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

In certain embodiments, an anti-cancer agent other than a c-Met inhibitor is a tumor cell pro-apoptotic or apoptosis-stimulating agent.

In certain embodiments, an anti-cancer agent other than a c-Met inhibitor is a signal transduction inhibitor. Signal transduction inhibitors include, for example: erbB2 receptor inhibitors, such as organic molecules, or antibodies that bind to the erbB2 receptor, for example, trastuzumab (e.g. Herceptin®); inhibitors of other protein tyrosine-kinases, e.g. imitinib (e.g. Gleevec®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer); GW-282974 (Glaxo Wellcome plc); monoclonal antibodies such as AR-209 (Aronex Pharmaceuticals Inc. of The Woodlands, Tex., USA) and 2B-1 (Chiron); and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

In certain embodiments, an anti-cancer agent other than a c-Met inhibitor is a cancer immunotherapy agent, such as an antibody specifically binding to an immune checkpoint. Immune checkpoints include, for example: A2AR, B7.1, B7.2, B7-H2, B7-H3, B7-H4, B7-H6, BTLA, CD48, CD160, CD244, CTLA-4, ICOS, LAG-3, LILRB1, LILRB2, LILRB4, OX40, PD-1, PD-L1, PD-L2, SIRPalpha (CD47), TIGIT, TIM-3, TIM-1, TIM-4, and VISTA.

In certain embodiments, an anti-cancer agent other than a c-Met inhibitor is an anti-proliferative agent. Anti-proliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

In certain embodiments, an anti-cancer agent other than a c-Met inhibitor is an cytotoxic agent. Cytotoxic agents according to the present invention include DNA damaging agents, antimetabolites, anti-microtubule agents, antibiotic agents, etc. DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof. Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof. Anti-microtubule agents include without limitation vinca alkaloids, paclitaxel (Taxol®), docetaxel (Taxotere®), and ixabepilone (Ixempra®). Antibiotic agents include without limitation actinomycin, anthracyclines, valrubicin, epirubicin, bleomycin, plicamycin, mitomycin, pharmaceutically acceptable salts thereof, prodrugs, and combinations thereof.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

This example illustrates that a cancer patient having overexpression of both HGF and c-Met was responsive to c-Met inhibitor.

### Materials and Methods

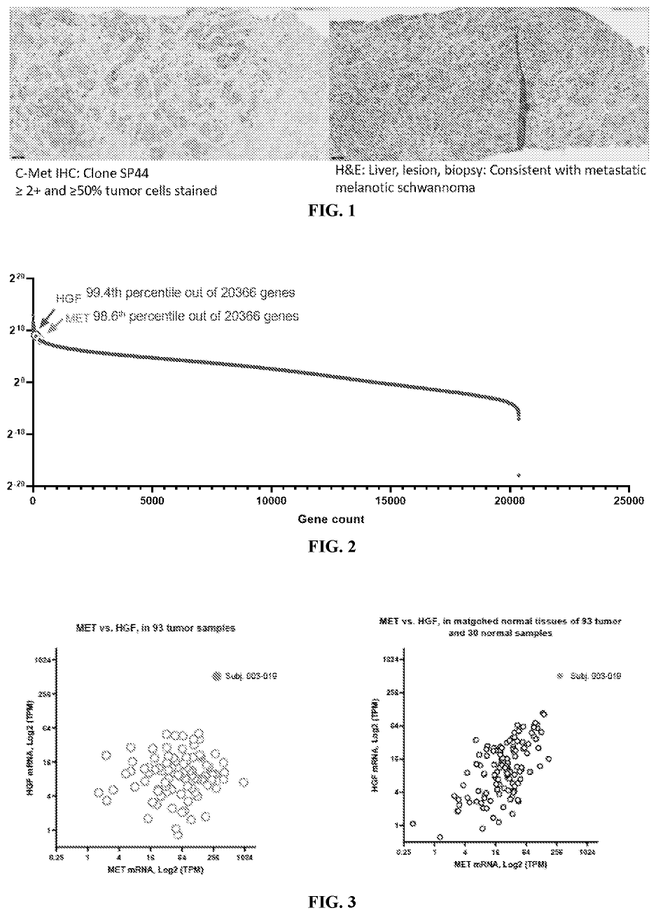

A Patient (003-019) was diagnosed with metastatic schwannoma and consented to be enrolled into a clinical trial for APL-101, a selective type 1b c-Met kinase inhibitor. The clinical trial was an open-label Phase 1 study to assess the safety and tolerability of APL-101, determine the recommended Phase 2 dose (RP2D) and dose limiting toxicities, and to obtain preliminary efficacy in subjects with c-Met dysregulated advanced solid tumors. The patient was enrolled based on high level of c-Met protein expression as detected by a c-Met immunohistochemistry (IHC) analysis of patient's tumor tissue sample at Caris Life Sciences (4610 S 44th Place, Phoenix, AZ, 85040) (FIG. 1). The Phase 1 study was completed with 17 subjects enrolled and treated with four escalating dose groups of 100-mg, 200-mg, 300-mg, and 400-mg per day, orally administered twice daily (BID). Based on the safety and PK results, the RP2D was determined to be 400-mg daily by the BID schedule. Patient 003-019 was treated with 300-mg APL-101 by the BID for a duration of 499 days (15.8 months). The patient's tumor sample was further analyzed by the MI Transcriptome sequencing assay at Caris Life Science to profile for genetic alterations and aberrant gene expressions carried by the patient's cancer at the transcript level covering the whole-transcriptome. The patient blood samples were collected at baseline and treatment termination to profile for genetic alterations at the DNA level in circulating tumor DNA (ctDNA) by the ArcherDx LiquidPlex at Archer Clinical Service (15000 W 6th Ave, Suite 150, Golden, CO 80401).

### Results

Patient 003-019 disease was assessed by radiographic evaluation based on RECIST criteria 1.1 according to protocol-defined schedule of every two dosing cycles. The patient first experienced disease control after 2 dosing cycles of APL-101 treatment, continuously benefited from the treatment with stable disease for 8 dose cycles of APL-101 and then experienced partial response after 10 dosing cycles. The patient continued to respond to APL-101 until disease progression at the end of the treatment. The patient experienced a duration of clinical benefits from APL-101 for 499 days (15.8 months).

To uncover potential predictive biomarkers associated with the patient's clinical response to APL-101 treatment, the patient's tumor and blood samples were analyzed to profile for genetic alterations captured by NGS of ctDNA and RNA transcripts of whole-transcriptome. The ArcherDx LiquidPlex ctDNA panel covers 100% of the MET genomic region and known hot spots of 28 other driver oncogenes including AKT1, ALK, AR, BRAF, CDK6, CTNNB1, EGFR, ERBB2, ERBB3, ESR1, FGFR1, FGFR2, FGFR3, HRAS, IDH1, IDH2, KIT KRAS, MAP2K1, NRAS, NTRK1, NTRK2, NTRK3, PDGFRA, PIK3CA, RET, ROS1, and TP53 for detection of aberrant variants and copy number variation. The Caris MI transcriptome test covers for the entire transcriptome for detection of gene expressions and aberrant variants on transcripts.

The ctDNA analysis of blood samples at pre-dose baseline and treatment termination did not detect known actionable driver oncogenic variants (Table 1.)

TABLE 1
No known oncogenic driver variants detected in patient 003-019 blood ctDNA

| | | | | | Clinical |
|---|---|---|---|---|---|
| Time point | Gene | Protein variant | DNA variant | COSMICID | Significance |
| Baseline | ESR1 | Gln502Pro | c.1505A > C | Not defined | unknown |
| | ESR1 | Gln500 deletion | c.1491_1493delGCA | Not defined | unknown |
| | TP53 | Not applicable | c.97-2A > C | Not defined | unknown |
| Treatment | IDH2 | Phe148Tyr | c.443T > A | Not defined | unknown |
| termination | NRAS | Asn116Asp | c.346A > G | Not defined | unknown |
| | NTRK1 | His571Leu | c.1712A > T | Not defined | unknown |

Whole transcriptome profiling results didn't detect known actionable driver oncogenic variants at the transcript level. However, the gene expression levels of both MET and HGF (measured as TPM) were identified to be aberrantly high, ranked in 99.4% and 98.6% percentiles respectively out of 20366 genes with transcript levels above the detection limit as shown in FIG. 2. When compared to a multi-tumor multi-tissue validation set, the HGF and c-Met expression level in the subject 003-019 are significantly higher than a dynamic range surveyed by a 30 tumor types and matched tissue types validation set of 93 unique samples (FIG. 3). These results supported a likely method to identify patients carrying wild type c-MET gene with aberrantly high levels of c-MET and HGF co-occurring gene expressions who may benefit from a c-Met inhibitor as exemplified by APL-101.

### Example 2

This example illustrates a method of identifying cancer patients, tumor cell lines, or patient-derived tumor models (PDX) carrying wild type MET with high-level of MET and HGF co-expression by RNA-sequencing analysis of gene expression of a select set of commonly aberrant genes across a broad spectrum of cancer types occurring in real-world. This example further validates whether cancer cells identified by such MET and HGF biomarker method are likely to be dependent on c-MET oncogenic pathway, respond to and benefit from treatment with c-Met inhibitors including APL-101.

### Materials and Methods

Forty commonly altered genes in all cancer patients with NGS profiling in the Genospace (Sarah Cannon Real-world evidence (RWE) cancer genomics database) are assembled to be a cancer gene-signature set for MET/HGF co-expression biomarker determination. The forty genes are: ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK.

The gene expression levels of each gene in the cancer-signature set are determined by RNA-sequencing of the transcriptome. The percentiles of the MET and HGF expression in this gene set are determined by rank-order (percentiles) of the gene expression levels as quantified by the units of a given RNA-sequencing method (for example, TPM or FPKM (fragments per kilobase of exon per million mapped fragments)).

Further, co-occurrence of key oncogenic driver mutations is examined by DNA-based exome-sequencing. The key genes for driver mutation analysis are MET, KRAS, BRAF, EGFR, ERBB2, ERBB3, PIK3CA.

To develop the relationship between MET/HGF co-expression biomarkers and response to treatment with c-MET inhibitor(s), tumor cell line xenograft models (XenoBase®, Crown Bioscience, CA) and PDX tumor models (HuBase®, Crown Bioscience, CA) were screened to identify tumor models carrying wild type MET with different levels of MET/HGF co-expression and different co-occurring oncogenic driver mutations. In vivo anti-tumor effects of APL-101 and selected c-MET inhibitors including an FDA approved c-Met inhibitor capmatinib were investigated in such models.

### Results

One tumor cell line xenograft model, two PDX tumor models were identified from XenoBase® and HuBase® to be carrying wild type MET with high to moderate of MET/HGF co-expression and were selected for in vivo anti-tumor evaluation. The histological characteristics and MET and HGF expression levels among the 40-cancer gene-signature genes are presented in Tables 2 and 3.

TABLE 2
Tumor models selected for MET/HGF
biomarker proof-of-concept study

| Model Type | Model Name | Species | Cancer Type |
|---|---|---|---|
| Xenograft | KP4 | Human | Pancreatic ductal adenocarcinoma |
| PDX | SA4097 | Human | Sarcoma (Western patient) |
| | SA10199 | Human | Synovial Sarcoma (Asian patient) |

TABLE 3
MET/HGF expression levels in the 40-cancer gene-
signature in selected tumor model by RNA sequencing

Gene Transcript Expression Level: Log2(FPKM)

| Gene | SA10199 | KP4 | SA4097 |
|---|---|---|---|
| ABL1 | 4.677 | 5.559 | 4.862 |
| ALK | −2.000 | −2.000 | −1.366 |
| ATM | 4.061 | 5.305 | 2.369 |
| ATR | 3.887 | 4.390 | 3.663 |
| AXL | 3.457 | 4.180 | 6.318 |
| BAP1 | 5.972 | 5.470 | 4.742 |
| BRAF | 2.701 | 3.998 | 2.998 |
| BRCA1 | 3.237 | 4.830 | 3.408 |
| BRCA2 | 3.030 | 2.245 | 2.047 |
| CHEK2 | 3.877 | 4.614 | 3.328 |
| DDR2 | 4.804 | −0.974 | 4.048 |
| EGFR | 5.378 | 3.532 | 2.279 |
| ERBB2 | 4.125 | 5.338 | 3.700 |
| ERBB4 | −0.599 | −0.656 | −2.000 |
| FGFR1 | 8.953 | 6.298 | 6.222 |
| FGFR2 | −0.373 | 0.847 | −2.000 |
| FGFR3 | 2.382 | 3.230 | −2.000 |
| FLT1 | −0.095 | −2.000 | −2.000 |
| FLT4 | 2.004 | −1.991 | −2.000 |
| HGF | 11.226 | 9.254 | 4.370 |
| HRAS | 5.349 | 5.673 | 6.856 |
| KDR | 0.102 | −2.000 | −2.000 |
| KIT | −2.000 | −1.122 | −2.000 |
| KRAS | 4.159 | 4.387 | 3.369 |
| MERTK | 3.419 | 3.084 | −2.000 |
| MET | 9.193 | 7.113 | 8.855 |
| MYC | 5.477 | 6.448 | 5.828 |
| NF1 | 3.833 | 5.327 | 3.316 |
| NRAS | 4.440 | 4.887 | 4.642 |
| NTRK1 | 1.456 | −1.542 | −2.000 |
| NTRK2 | −1.340 | −2.000 | 2.102 |
| NTRK3 | −1.566 | −2.000 | 1.353 |
| PDGFRA | 4.211 | 3.084 | −1.264 |
| PDGFRB | 7.520 | −2.000 | 3.319 |
| PIK3CA | 3.513 | 3.878 | 4.353 |
| PTEN | 3.028 | 5.104 | 5.382 |
| RAF1 | 5.834 | 6.047 | 4.546 |
| RET | −1.565 | −1.310 | −2.000 |
| ROS1 | −2.000 | −2.000 | −2.000 |
| TEK | −0.160 | −1.278 | 3.845 |

The co-occurrence of key oncogenic driver mutations is examined by DNA-based exome-sequencing in these three tumor models (Table 4). All three models carry wild type MET. KP4 carries a known oncogenic KRAS mutations G12D and wild type for the other five key oncogenic driver genes, whereas the other two models are wild type for all six key oncogene driver genes.

TABLE 4
Key oncogenic driver mutations by exome sequencing

| Gene | SA10199 | SA4097 | KP4 |
|---|---|---|---|
| MET | Negative | Negative | Negative |
| KRAS | Negative | Negative | G12D |
| BRAF | Negative | Negative | Negative |
| EGFR | Negative | Negative | Negative |
| ERBB2 | Negative | Negative | Negative |
| ERBB3 | Negative | Negative | Negative |
| PIK3CA | Negative | Negative | Negative |

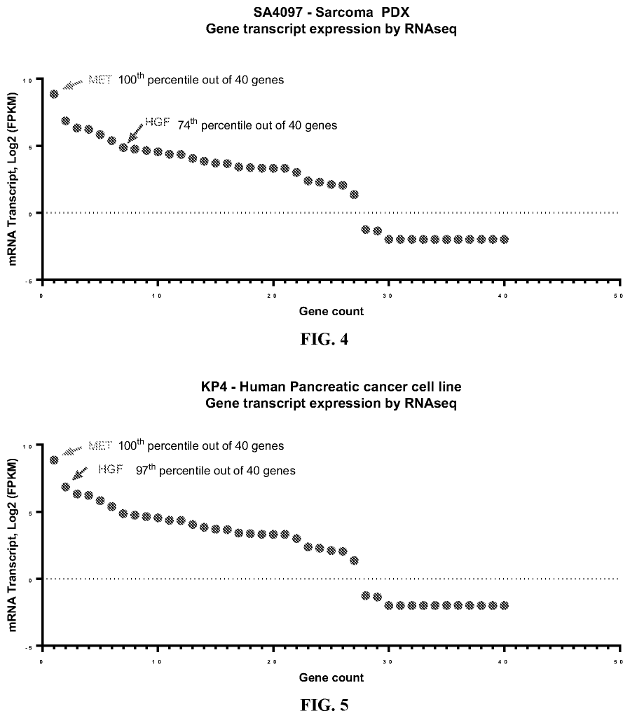

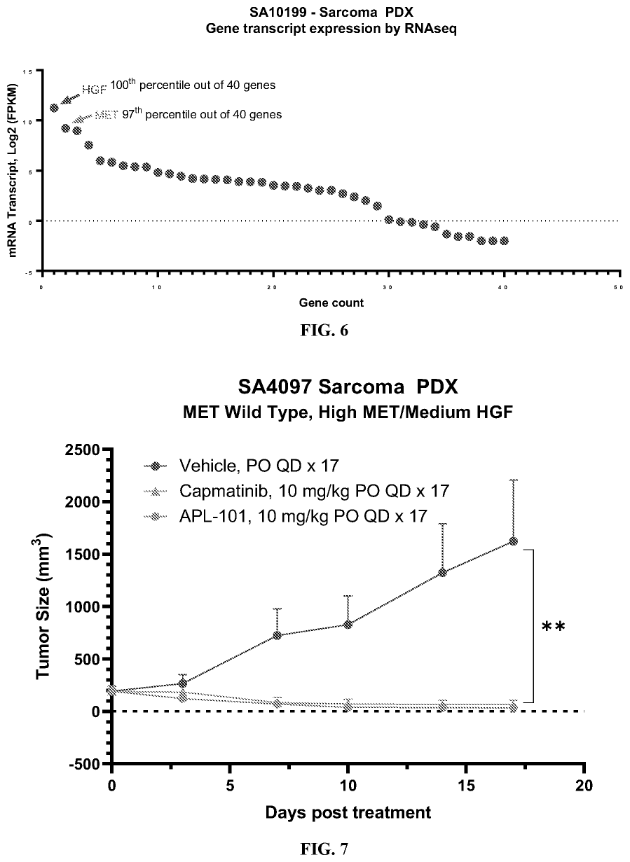

The percentiles of the MET and HGF gene expression ranking among the 40-cancer gene-signatures are presented in FIGS. 4, 5, and 6.

The percentiles of MET and HGF are the highest in KP4 and SA10199. However, in SA4097, although the percentile of MET is the highest in SA4097, the percentile of HGF is moderate ranking 74^{th} percentile.

To understand whether a lower threshold of HGF when co-expressed with MET may confer dependence to c-Met oncogenic pathway, SA4097-bearing mice were treated with APL-101 and an approved c-Met inhibitor capmatinib in vivo at 10 mg/kg QD which was shown to be a therapeutically active dose for both inhibitors in pharmacology studies. APL-101 and capmatinib demonstrated strong tumor growth inhibition of SA4097 (FIG. 7). This result further supports the initial biomarker hypothesis generated from the clinical case story of patient 003-019 as presented in Example 1 and extend the understanding what thresholds of co-expression levels of MET and HGF may confer a treatment response to c-MET inhibitor as exemplified by APL-101 and capmatinib.

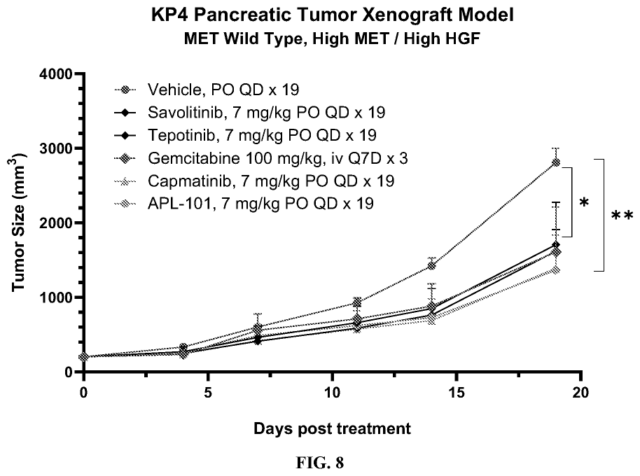

To understand whether a co-occurring oncogenic driver mutation confers upfront resistance to tumors carrying wild type MET with high-level of both MET and HGF, mice bearing KP4 tumor xenografts were treated with APL-101 and three additional approved c-Met inhibitors capmatinib, tepotinib and savolitinib in vivo at 7 mg/kg QD, which was also shown to be a therapeutically active dose for these inhibitors in pharmacology studies (FIG. 8). All c-Met inhibitors showed a partial tumor growth inhibition, with APL-101 and capmatinib showing numerically stronger anti-tumor effects than the other two c-Met inhibitors. This result suggests that a co-occurring oncogenic mutation such as KRAS may attenuate the anti-tumor effects of a c-Met inhibitor on a tumor that are otherwise likely dependent on the MET pathway due to high levels of MET and HGF in MET wild type genetic background.

What is claimed is:

1. A method of treating a subject having a cancer, the method comprising:
detecting substantially all RNA transcripts expressed in a sample from the subject, thereby measuring expression level of each gene in a whole transcriptome of the sample;
determining that the expression level of HGF is greater than at least 95% of all genes in the whole transcriptome;
determining that the expression level of c-Met is greater than at least 95% of all genes in the whole transcriptome; and
administering to the subject a therapeutic effective amount of a c-Met inhibitor.

2. The method of claim 1, wherein the expression level of HGF or c-Met is greater than at least 96%, 97%, 98%, or 99% of all genes in the whole transcriptome.

3. The method of claim 1, comprising ranking the expression level of each gene in the whole transcriptome before determining that the expression level of HGF or c-Met is greater than a threshold percentage of all genes in the whole transcriptome.

4. The method of claim 1, wherein the sample does not have a mutation in c-Met gene.

5. The method of claim 1, wherein the sample does not have an oncogenic mutation in c-Met gene.

6. The method of claim 1, wherein the cancer is selected from the groups consisting of a lung cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

7. The method of claim 1, wherein the cancer is a non-small cell lung cancer (NSCLC), renal cell carcinoma or hepatocellular carcinoma.

8. The method of claim 1, wherein the sample is tissue or blood.

9. The method of claim 1, wherein the RNA transcripts are detected using high throughput sequencing.

10. The method of claim 1, wherein the c-Met inhibitor is selected from the group consisting of crizotinib, cabozantinib, APL-101 (CBT-101, PLB1001, bozitinib), SU11274, PHA665752, K252a, PF-2341066, AM7, JNJ-38877605, PF-04217903, MK2461, GSK1363089 (XL880, foretinib), AMG458, tivantinib (ARQ197), INCB28060 (INC280, capmatinib), E7050, BMS-777607, savolitinib (volitinib), tepotinib, HQP-8361, merestinib, ARGX-111, onartuzumab, rilotumumab, emibetuzumab, and XL184.

11. The method of 1, wherein the c-Met inhibitor is a compound of the following formula 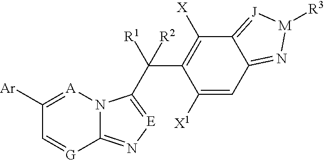
wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl.

12. The method of claim 11, wherein the compound has the following formula

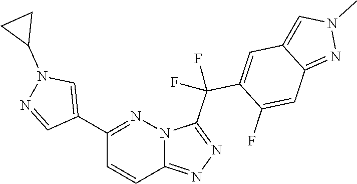 [SMILES: Cn1cc2cc(C(F)(F)c3nnc4ccc(-c5cnn(C6CC6)c5)nn34)c(F)cc2n1]

13. The method of claim 1, wherein the c-Met inhibitor is an anti-c-Met antibody.

14. A method for identifying a subject having cancer as likely to respond to treatment with a c-Met inhibitor, the method comprising:
obtaining a sample from the subject;
detecting substantially all RNA transcripts expressed in the sample, thereby measuring expression level of each gene in a whole transcriptome of the sample;
determining that the expression level of HGF is greater than at least 95% of the whole transcriptome;
determining that the expression level of c-Met is greater than at least 95% of the whole transcriptome; and
determining that the subject is likely to respond to the treatment with a c-Met inhibitor.

15. The method of claim 14, further comprising administering to the subject a therapeutic effective amount of the c-Met inhibitor.

16. A method of treating a subject having a cancer, the method comprising:
detecting RNA transcripts of a set of biomarker genes expressed in a sample from the subject, thereby measuring expression level of each gene in the set of biomarker genes of the sample, wherein the set of biomarker genes comprises ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK;
determining that the expression level of HGF is greater than at least 70% of all other genes in the set of biomarker genes;
determining that the expression level of c-Met is greater than at least 95% of all other genes in the set of biomarker genes; and
administering to the subject a therapeutic effective amount of a c-Met inhibitor.

17. The method of claim 16, wherein the expression level of HGF is greater than at least 75%, 80%, 85%, 90%, 95%, 96%, 97%, 98%, 99% or 100% of all other genes in the set of biomarker genes or the expression level of c-Met is greater than at least 96%, 97%, 98%, 99% or 100% of all other genes in the set of biomarker genes.

18. The method of claim 16, comprising ranking the expression level of each gene in the set of biomarker genes before determining that the expression level of HGF or c-Met is greater than a threshold percentage of all other genes in the set of biomarker genes.

19. The method of claim 16, wherein the sample does not have a mutation in c-Met gene.

20. The method of claim 16, wherein the sample does not have an oncogenic mutation in c-Met gene.

21. The method of claim 16, wherein the cancer is selected from the groups consisting of a lung cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

22. The method of claim 16, wherein the cancer is a non-small cell lung cancer (NSCLC), renal cell carcinoma or hepatocellular carcinoma.

23. The method of claim 16, wherein the sample is tissue or blood.

24. The method of claim 16, wherein the RNA transcripts are detected using high throughput sequencing.

25. The method of claim 16, wherein the c-Met inhibitor is selected from the group consisting of crizotinib, cabozantinib, APL-101 (CBT-101, PLB1001, bozitinib), SU11274, PHA665752, K252a, PF-2341066, AM7, JNJ-38877605, PF-04217903, MK2461, GSK1363089 (XL880, foretinib), AMG458, tivantinib (ARQ197), INCB28060 (INC280, capmatinib), E7050, BMS-777607, savolitinib (volitinib), tepotinib, HQP-8361, merestinib, ARGX-111, onartuzumab, rilotumumab, emibetuzumab, and XL184.

26. The method of 16, wherein the c-Met inhibitor comprises a compound of the following formula 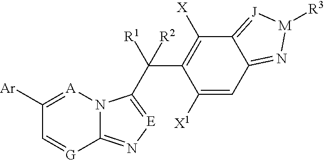
wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or CH;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl.

27. The method of claim 26, wherein the compound has the following formula

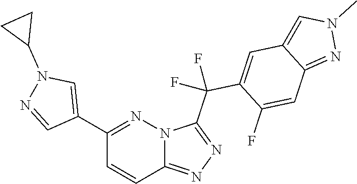 [SMILES: Cn1cc2cc(C(F)(F)c3nnc4ccc(-c5cnn(C6CC6)c5)nn34)c(F)cc2n1]

28. The method of claim 16, wherein the c-Met inhibitor is an anti-c-Met antibody.

29. A method for identifying a subject having cancer as likely to respond to treatment with a c-Met inhibitor, the method comprising:
obtaining a sample from the subject;
detecting RNA transcripts of a set of biomarker genes expressed in the sample, thereby measuring expression level of each gene in the set of biomarker genes of the sample, wherein the set of biomarker genes comprises ABL1, ALK, ATM, ATR, AXL, BAP1, BRAF, BRCA1, BRCA2, CHEK2, DDR2, EGFR, ERBB2, ERBB4, FGFR1, FGFR2, FGFR3, FLT1, FLT4, HGF, HRAS, KDR, KIT, KRAS, MERTK, MET, MYC, NF1, NRAS, NTRK1, NTRK2, NTRK3, PDGFRA, PDGFRB, PIK3CA, PTEN, RAF1, RET, ROS1, TEK;
determining that the expression level of HGF is greater than at least 70% of the other genes in the set of biomarker genes;
determining that the expression level of c-Met is greater than at least 95% of the other genes in the set of biomarker genes; and
determining that the subject is likely to respond to the treatment with a c-Met inhibitor.

30. The method of claim 26, further comprising administering to the subject a therapeutic effective amount of the c-Met inhibitor.
